(19) United States
(12) Patent Application Publication
(10) Pub. No.: US 2005/0209294 A1
(43) Pub. Date: Sep. 22, 2005

(54) Process for producing 4-(1H-1,2,4-triazol-1-ylmethyl)benzonitrile

(76) Inventors: Lalit Kumar Wadhwa, Panchkula (IN); Rahul Saxena, Chandigarh (IN)

Correspondence Address: CHRISTIE, PARKER & HALE, LLP, PO BOX 7068, PASADENA, CA, 91109-7068, US

(21) Appl. No.: 10/802,541

(22) Filed: Mar. 17, 2004

(51) Int. Cl. A61K 31/4196; C07D 249/08

(52) U.S. Cl. 514/383; 548/269.4

(57) ABSTRACT

The invention discloses an improved process for producing 4-(1H-1,2,4-triazol-1-ylmethyl)benzonitrile of Formula (Structure 2), an intermediate used in the manufacture of 4,4′-[1H-1,2,4-triazol-1-ylmethylene] bisbenzonitrile (Letrozole), the process comprising of reacting salt of 1,2,4-triazole of Formula (Structure 4) with α-halo substituted tolunitrile of Formula (Structure 3) in presence of dimethylformamide, wherein the X represents alkali metals selected from a group of Li, Na, or K, preferably Na and Y represents a halogen group selected from Cl, Br or I, preferably Br.

### FIELD OF THE INVENTION

This invention relates to an improved process for producing 4-(1H-1,2,4-triazol-1-ylmethyl)benzonitrile an intermediate used in the manufacture of 4,4′-[1H-1,2,4-triazol-1-ylmethylene]bisbenzonitrile (Letrozole).

### BACKGROUND OF THE INVENTION

4,4′-[1H-1,2,4-triazol-1-ylmethylene]bisbenzonitrile is the compound of Formula (Structure 1). It is a potent aromatase inhibitor to inhibit estrogen biosynthesis which is effective in the treatment of hormone-dependent breast cancer in postmenopausal women. Estrogen deprivation is most specifically achieved using inhibitors, which block the last stage in the biosynthetic sequence, i.e., the conversion of androgens to estrogens by the aromatase enzyme. Similarly, experimental studies demonstrate that letrozole substantially inhibits aromatase activity in both malignant and nonmalignant breast tissues, and markedly suppresses endogenous estrogens within the breast cancers.

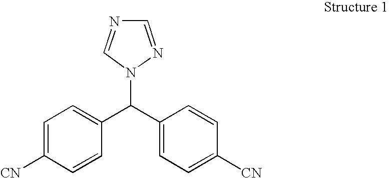

Several processes are known for the preparation of Letrozole. The known process includes a process for producing Letrozole employing the intermediate namely 4-(1H-1,2,4-triazol- 1-ylmethyl)benzonitrile.

U.S. Pat. No. 4,978,672 and U.S. Pat. No. 4,937,250 to Bowman, et al., disclose a process for preparation of α-heterocyclic substituted tolunitrile used as an aromatase-inhibitor wherein the process comprises of mixing a solution of α-bromo-4-tolunitrile in dichloromethane with imidazole. The mixture is stirred at ambient temperature for 15 hours and then diluted with water (1000 ml). Any undissolved solid is removed by filtration and the separated organic solution is then repeatedly washed with water (5×200 ml) to remove excess imidazole, and then dried (MgSO₄). The crude product obtained upon evaporation of the solvent can be purified by trituration with cold diethyl ether (200 ml) to obtain 4-(1-imidazolylmethyl)benzonitrile, which is later reacted with 4-fluorobenzonitrile and potassium tertiary butoxide in presence of dimethylformamide to afford the title compound.

U.S. Pat. No. 5,473,078 to Bowman, et al., discloses a method of preparation of 4-(1H-1,2,4-triazol-1-ylmethyl)benzonitrile, an intermediate used in the preparation of Letrozole. The method discloses refluxing a solution containing α-bromo-4-tolunitrile and 1,2,4-triazole in a mixture of chloroform and acetonitrile with stirring for 15 hours. The solution is cooled and washed with 3% aqueous sodium bicarbonate and the organic solution is then dried and evaporated. The residue is chromatographed on silica gel and elution with chloroform/isopropanol affords 4-(1H-1,2,4-triazol-1-ylmethyl)benzonitrile, which is later reacted with 4-fluorobenzonitrile and potassium tertiary butoxide in presence of dimethyl formamide to afford the title compound.

The known process suffers from a variety of disadvantages including the fact that during the course of the synthesis of 4-(1H-1,2,4-triazol-1-ylmethyl)benzonitrile, the non selective reaction also yields the unwanted isomer named 4-(1H-1,3,4-triazol-1-ylmethyl)benzonitrile in about 50% ratio. This requires separating the desired intermediate through column chromatography. This is extremely disadvantageous in large scale productions of the title compound.

The present invention discloses an alternative route of synthesis of this intermediate by the reaction of suitable salt of 1,2,4-triazole with α-bromo-4-tolunitrile to afford desired intermediate with >96% selectivity, thereby circumventing the tedious column chromatography procedure. The said intermediate is then converted to Letrozole of USP quality, without chromatographic separation, following conventional procedure.

The invention disclosed herein demonstrates economically viable selective synthesis of advanced intermediate for Letrozole, circumvention of column chromatography procedure allowing the process to become industrial friendly for commercial scale and also better time cycle for the reaction.

### SUMMARY OF THE INVENTION

It is a principal aspect of the present invention to provide for an improved process for producing 4-(1H-1,2,4-triazol-1-ylmethyl)benzonitrile, which is a useful intermediate in producing Letrozole.

In one preferred embodiment, disclosed herein is a process for producing 4-(1H-1,2,4-triazol-1-ylmethyl)benzonitrile, by reacting alkali metal salt of 1,2,4-triazole with α-halo substituted tolunitrile in presence of dimethylformamide.

In another aspect, the present invention provides for an improved process for producing 4-(1H-1,2,4-triazol-1-ylmethyl)benzonitrile wherein the process obviates the use of expensive column chromatography and makes it cost efficient and time efficient. The 4-(1H-1,2,4-triazol-1-ylmethyl)benzonitrile thus produced is then used in the manufacture of USP quality 4,4′-[1 H-1,2,4-triazol-1-ylmethylene]bisbenzonitrile (Letrozole) employing conventional procedures.

In one another preferred embodiment, the present invention provides for an improved process for producing 4-(1H-1,2,4-triazol-1-ylmethyl)benzonitrile the process comprising, producing 4-(1H-1,2,4-triazol-1-ylmethyl)benzonitrile regioselectively, using α-bromo-4-tolunitrile with sodium salt of 1H-1,2,4-triazole in the presence of dimethylformamide. The 4-(1H-1,2,4-triazol-1-ylmethyl)benzonitrile thus produced is then used in the manufacture of USP quality 4,4′-[1H-1,2,4-triazol-1-ylmethylene]bisbenzonitrile (Letrozole) employing conventional procedures.

### DETAILED DESCRIPTION OF THE INVENTION

The disclosed embodiment of the present invention deals with a process for the preparation of 4-(1H-1,2,4-triazol-1-ylmethyl)benzonitrile of Formula (Structure 2) that has advantages over prior art processes in that it avoids formation of undesired product, uses less amount of solvents and eliminates undesired processing steps to make it comparatively time and cost effective process.

A process for producing 4-(1H-1,2,4-triazol-1-ylmethyl)benzonitrile of Formula (Structure 2) has been provided, the process comprising of reacting a salt of 1,2,4-triazole of Formula (Structure 4) with α-halo substituted tolunitrile of Formula (Structure 3) in presence of dimethylformamide. Aforesaid reaction is being carried out by charging dimethylformamide followed by salt of 1,2,4triazole at 25-30° C., adding a solution of α-halo substituted tolunitrile in dimethylformamide at 10° C., stirring the same for 2 hours at 10 to 15° C., thereafter adding demineralized water and extracting with dichloromethane, distilling out the organic layer and crystallizing the same from diisopropyl ether.

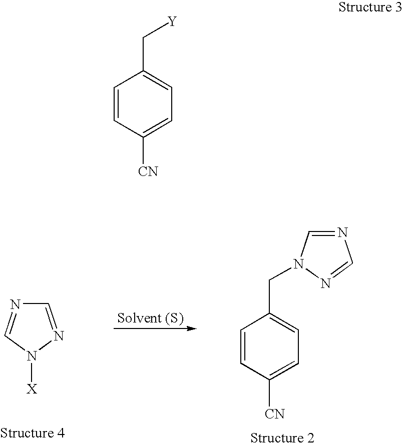

A process for producing 4-(1H-1,2,4-triazol-1-ylmethyl)benzonitrile of Formula (Structure 2), which is then converted to Letrozole of USP quality, without chromatographic separation, following conventional procedure. Aforesaid reaction is being carried out by reacting 4-(1H-1,2,4-triazol-1-ylmethyl)benzonitrile of Formula (Structure 2) with 4-fluorobenzonitrile and potassium tertiary butoxide to afford the title compound of Formula (Structure 1).

A process for producing 4-(1H-1,2,4-triazol-1-ylmethyl)benzonitrile of Formula (Structure 2) comprising of reacting a salt of 1,2,4-triazole of Formula (Structure 4) with α-halo substituted tolunitrile of Formula (Structure 3) in presence of dimethylformamide, wherein Y represents a halogen group selected from Cl, Br or I, preferably Br.

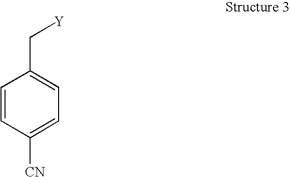

A process for producing 4-(1H-1,2,4-triazol-1-ylmethyl)benzonitrile of Formula (Structure 2), the process comprising of reacting salt of 1,2,4-triazole of Formula (Structure 4) with α-halo substituted tolunitrile of Formula (Structure 3) in presence of dimethylformamide, wherein X represents alkali metals selected from a group comprising Li, Na, or K, preferably Na.

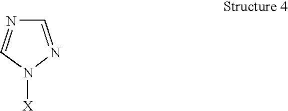

Preferred embodiments are further illustrated in the following example:

### EXAMPLE 1

To a solution of 98 g of sodium salt of 1,2,4 triazole in 100 cc of dimethylformamide (DMF) at 25-30° C., a solution of 100 g of 4-Bromomethyl benzonitrile in 250 cc of dimethylformamide at 10° C. was added over 30 minutes. After the completion of addition, the mixture was stirred at 10-15° C. for further two hours. DM water (800 cc) was added and the reaction mass was extracted twice with dichloromethane (300 cc). The combined organic layer was washed with water (2×100 cc), dried over sodium sulfate and was distilled off. The crude was crystallized from diisopropyl ether to afford 4-(1H-1,2,4,-triazol-1-ylmethyl)benzonitrile.

Certain modifications and improvements of the disclosed invention will occur to those skilled in the art without departing from the scope of invention, which is limited only by the appended claims.

1. A process for producing 4-(1H-1,2,4-triazol-1-ylmethyl)benzonitrile of Formula (Structure 2), the process comprising, reacting a salt of 1,2,4-triazole of Formula (Structure 4) with α-halo substituted tolunitrile of Formula (Structure 3) in the presence of a suitable solvent, wherein the reaction is carried out by charging in the solvent followed by addition of a salt of 1,2,4-triazole of Formula (Structure 4) at 25-30° C., adding a solution of α-halo substituted tolunitrile of Formula (Structure 3) in the solvent at 10° C.; stirring the same for 2 hours at 10 to 15° C.; adding demineralized water and extracting with dichloromethane; distilling out the organic layer; crystallizing the same using a crystallizing agent to obtain 4-(1H-1,2,4-triazol-l-ylmethyl)benzonitrile of Formula (Structure 2).

2. The process according to claim 1, wherein X represents an alkali metal selected from a group comprising Li, Na, or K.

3. The process according to claim 2, wherein X represents Na.

4. The process according to claim 1, wherein Y represents a halogen selected from Cl, Br or I.

5. The process according to claim 4, wherein Y represents Br.

6. The process according to claim 1, wherein the suitable solvent is tetrahydrofurane or dimethylformamide.

7. The process according to claim 6 wherein the preferred solvent is dimethylformamide.

8. The process according to claim 1, wherein the crystallizing agent is an organic solvent selected from a group comprising isopropyl alcohol, toluene or diisopropyl ether.

9. The process according to claim 8, wherein the preferred organic solvent is diisopropyl ether.

10. A process for producing 4-(1H-1,2,4-triazol-1-ylmethyl)benzonitrile of Formula (Structure 2), the process comprising:
charging dimethylformamide followed by addition of sodium salt of 1,2,4 triazole at 25-30° C.;
adding a solution of α-bromo-4-tolunitrile in dimethylformamide at 10° C.;
stirring the same for 2 hours at 10 to 15° C.;
adding demineralized water and extracting with dichloromethane;
distilling out the organic layer; and
crystallizing the same in diisopropyl ether to obtain 4-(1H-1,2,4-triazol-1-ylmethyl)benzonitrile of Formula (Structure 2).

11. The process according to claim 10 further comprising:
reacting 4-(1H-1,2,4-triazol-1-ylmethyl)benzonitrile of Formula (Structure 2), with 4-fluorobenzonitrile and potassium tertiary butoxide to produce 4,4′-[1H-1,2,4-triazol-1-ylmethylene]bisbenzonitrile of Formula (Structure 1).

12. A process for producing 4,4′-[1H-1,2,4-triazol-1-ylmethylene]bisbenzonitrile of Formula (Structure 1), the process comprising reacting 4-(1H-1,2,4-triazol-1-ylmethyl)benzonitrile of Formula (Structure 2) produced according to the processes recited in any of claims 1 through to 11, the process comprising, reacting 4-(1H-1,2,4-triazol-1-ylmethyl)benzonitrile of Formula (Structure 2) with 4-fluorobenzonitrile and potassium tertiary butoxide to produce 4,4′-[1H-1,2,4-triazol-1-ylmethylene]bisbenzonitrile (Letrozole) of Formula (Structure 1).
